(19) United States
(12) Patent Application Publication
(10) Pub. No.: US 2022/0015745 A1
(43) Pub. Date: Jan. 20, 2022

(54) METHOD FOR PROVIDING SERVICE FOR INFERTILITY AND A SERVER PERFORMING THE METHOD

(71) Applicant: Dable Co., Ltd., Daegu (KR)

(72) Inventor: Ji Yeong SOH, Seoul (KR)

(21) Appl. No.: 17/374,397

(22) Filed: Jul. 13, 2021

(30) Foreign Application Priority Data
Jul. 15, 2020 (KR) 10-2020-0087548

(51) Int. Cl. A61B 10/00; G06Q 10/02; G06Q 10/08

(52) U.S. Cl. CPC A61B 10/0012; G06Q 10/0833; G06Q 10/02; A61B 10/0045

(57) ABSTRACT

This application relates to a method of providing an infertility-associated service. In one aspect, the method includes receiving, from a user terminal, a fertility test request including user information and selection information. The method may also include selecting a specimen collection method according to the selection information and performing sampling scheduling for the selected specimen collection method. The method may further include receiving a result of a fertility test performed using a specimen collected according to the selected specimen collection method, and providing the received result of the fertility test to the user terminal.

### CROSS REFERENCE TO RELATED APPLICATION

The present application claims priority to Korean Patent Application No. 10-2020-0087548 filed on Jul. 15, 2020, the entire contents of which are incorporated herein for all purposes by this reference.

### BACKGROUND

### Technical Field

The present disclosure generally relates to a method for providing an infertility-associated service and a server performing the same.

### Description of the Related Technology

Recently, there has been a growing demand for treatment of infertility or infertility. Infertility refers to a case where a couple is not pregnant even if they have a normal married life for more than one year without contraception. There are many reasons for the infertility, but the infertility cause of 30% to 40% thereof is not known.

### SUMMARY

The present disclosure relates to a method for providing an infertility-associated service and a server performing the same. Another aspect of the present disclosure is to provide a computer-readable recording medium having recorded thereon a program for executing the method in a computer. The technical objectives to be achieved by the present embodiment are not limited to the above-described technical objects, and other technical objects may be inferred from the following embodiments.

An embodiment of the present disclosure provides a method of providing an infertility-associated service, including: receiving, from a user terminal, a fertility test request including user information and selection information; selecting a specimen collection method according to the selection information and performing sampling scheduling for the selected specimen collection method; receiving a result of a fertility test performed using a specimen collected according to the selected specimen collection method; and providing the received result of the fertility test to the user terminal.

In an embodiment, the specimen collection method may be any one of a specialist visit collection method, a test kit delivery method, and a hospital visit collection method.

In an embodiment of the present disclosure, the performing of the delivery scheduling for the selected specimen collection method may include, in a case where the test kit delivery method is selected, making a delivery company delivery reservation to deliver the test kit to a location of the user included in the user information, and making a delivery company collection reservation to collect a test kit including the obtained specimen at a location of a user.

In an embodiment of the present disclosure, the method may further include, in a case where the test kit delivery method is selected, providing the user terminal with a specimen collection method using the test kit.

In an embodiment, the delivery scheduling for the selected sample collection method may include: receiving a completion event in which delivery of the test kit to a location of a user is completed or collection of a specimen is completed according to an input of the user; and making a delivery company collection reservation within a preset period from a time when the completion event occurs.

An embodiment of the present disclosure provides a server including: a memory storing at least one program; a communication unit for transmitting data to and receiving data from a user terminal, a delivery company for delivering and collecting test kits, and an examination institution for performing a fertility test; and at least one processor that provides an infertility-associated service to a user by executing the at least one program, wherein the memory has instructions to execute, by the at least one processor, receiving, from the user terminal, a fertility test request including user information and selection information, selecting a specimen collection method according to the selection information and performing sampling scheduling for the selected specimen collection method, receiving, from the examination institution, a result of a fertility test performed using the collected specimen, and providing the received result of the fertility test to the user terminal.

An embodiment of the present disclosure provides a computer-readable recording medium having recorded thereon a program for executing the method described above in a computer.

According to the above-described objective solution of the present disclosure, the embodiments of the present disclosure provide a service capable of allowing users having difficulty due to infertility to in-person test at home, thereby increasing user convenience. In addition, the embodiments of the present disclosure can improve the fertility of the user by providing appropriate fertility management information according to the result of the fertility test on the user or providing information about the fertility preservation procedure.

### BRIEF DESCRIPTION OF THE DRAWINGS

FIG. 1 is a block diagram of a system for providing an infertility-associated service according to an embodiment of the present disclosure.

FIG. 2 is a flowchart illustrating that an infertility-associated service is provided among a user terminal, an infertility-associated service platform, a delivery company, and an examination institution, according to an embodiment.

FIG. 3A and FIG. 3B are diagrams to describe a method in which a user selects a fertility test method by using a user terminal.

FIG. 4 and FIG. 5 are diagrams for describing a process in which an infertility-associated service platform performs a collection reservation.

FIG. 6 is a flowchart illustrating a method of providing an infertility-associated service, according to an embodiment.

FIG. 7 is a block diagram of an infertility-associated service supply server according to an embodiment.

### DETAILED DESCRIPTION

In order to prevent such infertility and to ensure a healthy pregnancy plan, fertility tests that evaluate women's ovarian health and men's sperm conditions are becoming important. Since a patient needs to make a reservation and visit a hospital in person for a fertility test, mental stress related to infertility and inconvenience in the process of testing are present.

Hereinafter, embodiments of the present disclosure will be described in detail with reference to the accompanying drawings so that those skilled in the art can easily implement the present disclosure. However, the present disclosure may be implemented in various different forms and is not limited to the embodiments described herein. In the drawings, elements not related to the description are omitted in order to clearly describe the present invention, and like elements are denoted by like reference numerals throughout the specification.

Throughout the specification, when an element is “connected” to another element, the element may be “directly connected” or may be “electrically connected” with another element wither other element therebetween. In addition, when a certain element “includes” a certain component, this means that other components may be further included, rather than excluding other components, unless otherwise stated.

Hereinafter, the present disclosure will be described in detail with reference to the accompanying drawings.

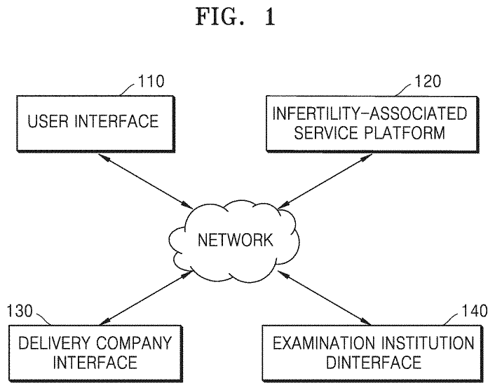

FIG. 1 is a block diagram of a system for providing an infertility-associated service according to an embodiment of the present disclosure.

Referring to FIG. 1, users who want to receive an infertility-associated service may access an infertility-associated service platform 120 through a user interface 110. In an embodiment, delivery companies that want to deliver test kits associated with infertility may access the infertility-associated service platform 120 through a delivery company interface 130. In addition, examination institutions performing the fertility test may access the infertility-associated service platform 120 through an examination institution interface 140. Hereinafter, users, delivery companies, and examination institutions include both domestic and foreign individuals and small and large companies.

The user interface 110, the delivery company interface 130 and the examination institution interface 140 may include a terminal, a portable terminal, a wearable terminal, a personal computer (PC), a notebook computer, a tablet, a smart phone, a smart watch, etc. The method of providing the infertility-associated service may be implemented by being installed in a terminal in the form of an application.

The infertility-associated service platform 120 may be connected, through a network, to one of the user interface 110, the delivery company interface 130, and the examination institution interface 140.

The network may include a local area network (LAN), a wide area network (WAN), a value-added network (VAN), a mobile radio communication network, a satellite communication network, and a combination thereof. In a comprehensive sense, the network refers to a data communication network that allows each network configuring entity shown in FIG. 1 to smoothly communicate with each other, and may include a wired Internet, a wireless Internet, and a mobile wireless communication network. The wireless communication may include, for example, wireless LAN (Wi-Fi), Bluetooth, Bluetooth low energy, Zigbee, Wi-Fi direct (WFD), ultra wideband (UWB), infrared data association (IrDA), and near field communication (NFC), but is not limited thereto.

The infertility-associated service platform 120 may correspond to an Internet website or a mobile phone application, but is not limited thereto.

The user inputs user information and selection information through the user interface 110. In this regard, the user information may include information about, for example, gender, age, height, weight, history of medical and reproductive diseases related to pregnancy, medication, surgery received in the past, delivery destination, and contact information. In the case of the female, the menstrual cycle, the start day of the first-time period, and the like may be further included.

Also, the selection information may be information about the method selected and input by the user who wants to receive the infertility-associated service. For example, the user may select a fertility test in which the user collects a specimen in person at home, or a specialist visits the user's home and collects a specimen, or the user may visit a hospital to receive the fertility test.

When the infertility-associated service platform 120 receives the test request of the user, the infertility-associated service platform 120 may perform scheduling for the fertility test according to the selection information of the user. The infertility-associated service platform 120 may provide user information about a delivery destination and a contact number to a delivery company according to the user's selection information, or may provide user information about pregnancy to an examination institution.

The delivery company may identify user information through the delivery company interface 130, deliver a test kit to the delivery destination, collect the test kit when the collection of a specimen is completed, and provide the same to an examination institution.

The examination institution may identify the user information through the examination institution interface 140, perform the fertility test by using the obtained specimen of the visiting user or the specimen of the delivered test kit, and provide the examination result to the infertility-associated service platform 120.

The infertility-associated service platform 120 may provide the received fertility test result to the user through the user interface 110.

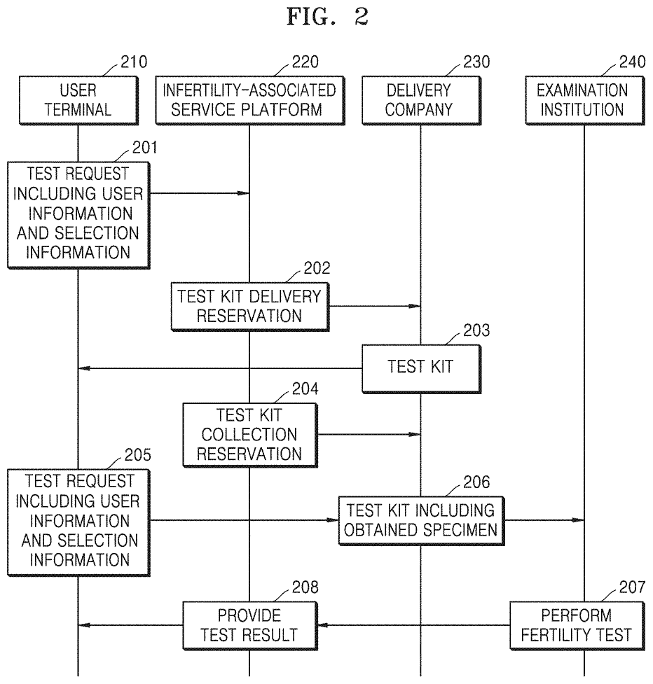

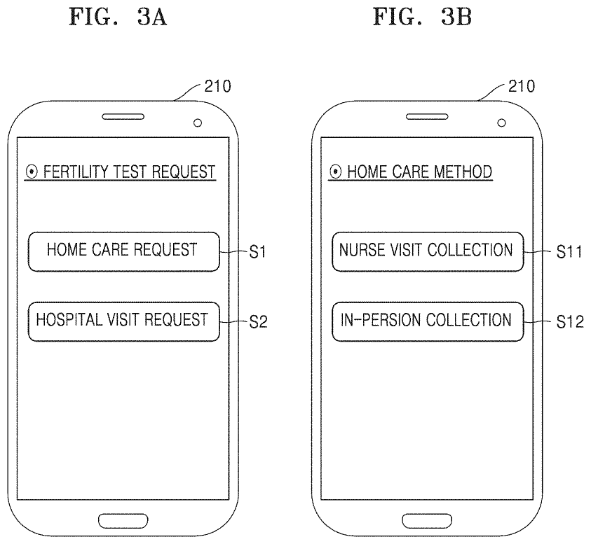

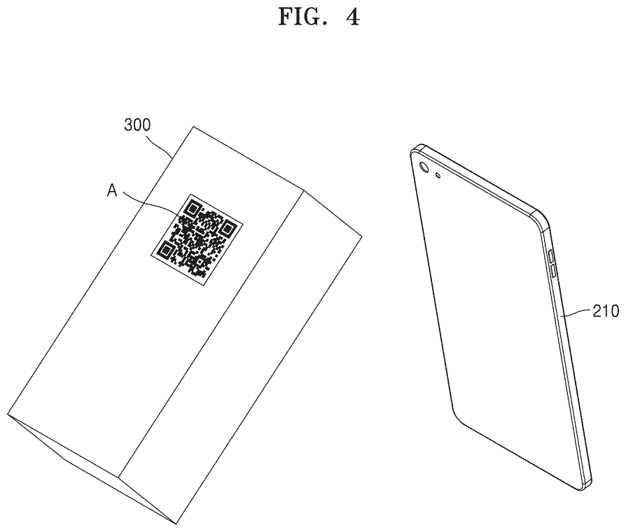

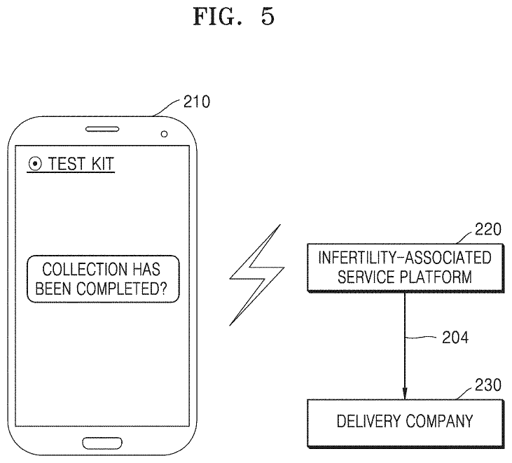

FIG. 2 is a flowchart illustrating that an infertility-associated service is provided among a user terminal, an infertility-associated service platform, a delivery company, and an examination institution, according to an embodiment, and FIG. 3A and FIG. 3B are diagrams to describe a method in which a user selects a fertility test method by using a user terminal. FIG. 4 and FIG. 5 are diagrams for describing a process in which an infertility-associated service platform performs a collection reservation.

Referring to FIGS. 2 to 5, in operation 201, a user terminal 210 may provide a test request including user information and selection information to an infertility-associated service platform 220. In an embodiment, the selection information may be information about an infertility-associated service supply method selected and input by the user. For example, the suer may select a fertility test in which the user collects a specimen in person at home, or a specialist visits the user's home and collects the specimen thereof, or the user may visit a hospital to receive the fertility test.

In an embodiment, as illustrated in FIG. 3A, the user may select any one of a home care application 51 for collecting a specimen at home through an interface of the user terminal 210 and a hospital visit application S for collecting a specimen through a hospital visit. When the user selects the home care application 51, as shown in FIG. 3B, the user may select one of a method S11 in which a specialist such as a nurse or the like visits home and collects the specimen and a method S12 in which the user receives a test kit by delivery and collects the specimen in person. The specimen collection method may be any one of a specialist visit collection method, a test kit delivery method, and a hospital visit collection method.

In operation 202, the infertility-associated service platform 220 may select a specimen collection method according to the selection information, and may perform scheduling for the selected specimen collection method. Hereinafter, for convenience of description, a method in which the user receives the test kit at home and collects the test kit in person will be described.

The infertility-associated service platform 220 may identify selection information selected by the user and then perform delivery scheduling for in-person collection. The infertility-associated service platform 220 may provide user information to a delivery company 230. The user information may include a delivery destination address, a recipient name, and a contact number.

In operation 203, when the user information is identified, the delivery company 230 may deliver a test kit to the user. The delivery company 230 may store a test kit for collecting an infertility-associated specimen and deliver the test kit to the user, or may deliver the test kit to the user in association with a test kit sales company.

Although not shown, when the test kit delivery method is selected, the infertility-associated service platform 220 may provide the user terminal with the specimen collecting method using the test kit. For example, in a case where the user is a woman and blood needs to be collected, a description about each step for collecting blood using the test kit may be provided through the user terminal. In addition, since the specimen is a biological sample, a storage environment including an appropriate temperature and the like may be required before collection. Accordingly, the infertility-associated service platform 220 may provide a user with a method of storing the obtained specimen through the user terminal.

In operation 204, the fertility-associated service platform 220 may perform a collection reservation for collecting a test kit including the obtained specimen. The infertility-associated service platform 220 may perform a collection reservation while providing user information to a delivery company so as to collect the test kit including the obtained specimen at the location of the user.

In an embodiment, the infertility-associated service platform 220 may receive a completion event in which the test kit is completely delivered to the location of the user or the collection of the specimen is completed according to the input of the user, and may perform a delivery company collection reservation within a preset period from the time when the completion event occurs.

For example, as illustrated in FIG. 4, when the user photographs an identification code A, such as a QR code, included in the packing material of a test kit 300 by using the user terminal before collecting the specimen, the user terminal 210 may provide the specimen collection completion event to the infertility-associated service platform 220. In an embodiment, as illustrated in FIG. 5, when the user selects collection completion by using the application of the user terminal after collecting the specimen, the user terminal 210 may provide the collection completion event to the infertility-associated service platform 220.

In an embodiment, the infertility-associated service platform 220 may receive a completion event which occurs when the package of the test kit is opened, and may perform a delivery company collection reservation within a preset period from the time when the completion event occurs.

In operation 205, the delivery company 230 may collect the test kit including the obtained specimen at the user's location.

In operation 206, the delivery company 230 may transfer the collected test kit to an examination institution 240.

In operation 207, the examination institution 240 may perform a fertility test of the user by using the test kit including the obtained specimen. In this regard, the fertility test may include, in the case of the female, a hormone test, an ovulation test, a postcoital test, a hysterosalpingography test, a laparoscopic test, a colposcopic test, an endometrial test, and the like, and in the case of a man, a sperm test, a hormone test, a vas deferens photography, an ultrasonography, and the like. Among these fertility tests, there are tests that can be performed only when a specialized institution such as a hospital must be visited, but a fertility test that is performed through collection of a specimen, such as, blood, sperm, and hormones can be performed without the user's visit to the hospital. For example, the fertility test may include a test using, as a specimen, a hormone which is used as an indicator for determining ovulation and fertility, and such a hormone may be an anti-mullerian hormone (AMH), a luteinizing hormone (LH), a follicle stimulating hormone (FSH), progesterone, estrogen, estradiol, and the like. The test kit may be a tool for collecting a specimen, and may be a tool for collecting a biological sample such as blood or a sperm.

In an embodiment, the test kit 300 illustrated in FIG. 4 may include a collection tool for collecting a specimen and an accommodation tool for accommodating the obtained specimen. In addition, the test kit 300 may include a storage member for storing the accommodation tool in which a specimen is accommodated. The storage member may include components for providing a storage environment suitable for a biological sample. For example, the storage member may include a temperature controller for controlling a temperature. In an embodiment, the storage member may include a driving unit that periodically rotates the accommodation tool so that blood is not solidified when the specimen is blood.

In operation 208, the infertility-associated service platform 220 may provide the examination result transferred by the examination institution to the user terminal 210. In this regard, since the user may want to visit a hospital and receive the result of the fertility test, the infertility-associated service platform 220 may allow the user to select how to receive the result when the result of the fertility test is transferred by the test institution.

When the user wants to visit the hospital and receive the result of fertility test, the infertility-associated service platform 220 may make a reservation for a visit to the hospital. In an embodiment, when the user wants to receive the test result at home, the result of the fertility test may be provided to the user terminal 210, together with description contents to help the user understand the same.

Although not shown, in a subsequent step, the infertility-associated service platform 220 may provide the fertility management information corresponding to the result of the fertility test. For example, the infertility-associated service platform 220 may provide a life guide for increasing the user's fertility, or may provide a service in which the user is connected to a specialist through a video meeting to receive a coaching related to the fertility in a uncontacted manner. In addition, the infertility-associated service platform 220 may perform a hospital visit reservation when the fertility is required to be preserved due to low fertility. As a method for preserving fertility, assisted reproductive surgery, egg freezing, and the like may be used.

In addition, the infertility-associated service platform 220 may receive a feedback regarding a result of performing the infertility care service, and may derive an infertility care service suitable for the user and provide the same again.

Embodiments of the present disclosure including the above-described operations provide a service capable of allowing users having difficulty due to infertility to in-person test at home, thereby increasing user convenience. In addition, the embodiments of the present disclosure can improve the fertility of the user by providing appropriate fertility management information according to the result of the fertility test on the user or providing information about the fertility preservation procedure.

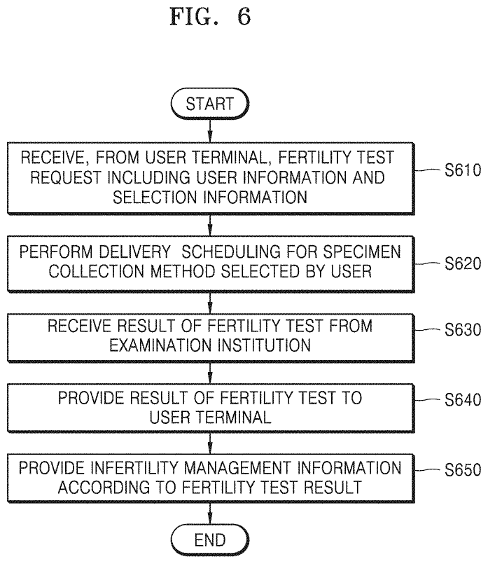

FIG. 6 is a flowchart illustrating a method of providing an infertility-related service, according to an embodiment.

In operation S610, an infertility-associated service supply server may receive, from the user terminal, a fertility test request including user information and selection information. In this regard, the user information may include information about, for example, gender, age, height, weight, history of medical and reproductive diseases related to pregnancy, medication, surgery received in the past, delivery destination, and contact information. In the case of the female, the menstrual cycle, the start day of the first-time period, and the like may be further included.

In an embodiment, the selection information may be information about an infertility-associated service supply method selected and input by the user. For example, a user may select a fertility test in which the user collects a specimen in person at home, or a specialist visits the user's home and collects the specimen thereof, or the user may visit a hospital to receive the fertility test.

In operation S620, the infertility-associated service supply server may perform sampling scheduling for the specimen collection method selected by the user. In this regard, the specimen collection method may be any one of a specialist visit collection method, a test kit delivery method, and a hospital visit collection method. The infertility-associated service supply server may perform a delivery scheduling when the user selects a test kit delivery method. However, the present embodiment is not limited thereto. In an embodiment, when the user selects a specialist visit collection method or a hospital visit collection method, the infertility-associated service supply server may perform a reservation scheduling.

In step S630, the infertility-associated service supply server may receive the result of the fertility test from the examination institution.

In step S640, the infertility-associated service supply server may provide, to the user terminal, the result of the fertility test received from the examination institution.

In step S650, the infertility-associated service supply server may provide infertility management information according to the fertility test result. In this regard, the infertility management information may include exercise information, eating habits information, sleep information, and the like, which are suitable for improving the user's fertility. Alternatively, the infertility management information may include treatment information about how to improve fertility. The treatment information may include information about a treatment device for improving the fertility of the user.

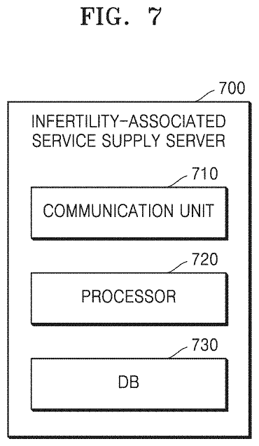

FIG. 7 is a block diagram of an infertility-associated service supply server according to an embodiment.

Referring to FIG. 7, an infertility-associated service supply server 700 may include a communication unit 710, a processor 720, and a DB 730. FIG. 7 illustrates only elements of the infertility-associated service supply server 700 which are associated with an embodiment. Therefore, it will be understood by those skilled in the art that other general-purpose elements may be further included in addition to the elements illustrated in FIG. 7.

The communication unit 710 may include one or more components for performing wired/wireless communication with users, delivery companies, and examination institutions. For example, the communicator 710 may include at least one of a local-area communicator (not shown), a mobile communicator (not shown), and a broadcast receiver (not shown).

The DB 730 is hardware for storing various data processed in the infertility-associated service supply server 700, and may store a program for processing and controlling the processor 720. The DB 730 may store various data necessary to provide infertility-associated service.

The DB 730 may include a random-access memory (RAM) such as a dynamic random-access memory (DRAM), a static random-access memory (SRAM), etc., a read-only memory (ROM), an electrically erasable programmable read-only memory (EEPROM), a CD-ROM, a Blu-ray or other optical disk storage, a hard disk drive (HDD), a solid-state drive (SSD), or a flash memory.

The processor 720 controls the overall operation of a infertility-associated service providing server 700. For example, the processor 720 may control an input unit (not shown), a display (not shown), the communicator 710, the DB 730, and the like by executing programs stored in the DB 730. The processor 720 may control an operation of the infertility-associated service providing server 700 by executing programs stored in the DB 730.

The processor 720 may be implemented using at least one of application specific integrated circuits (ASICs), digital signal processors (DSPs), digital signal processing devices (DSPDs), programmable logic devices (PLDs), field programmable gate arrays (FPGAs), controllers, micro-controllers, microprocessors, and electrical units for performing other functions.

The present embodiments may be implemented even in the form of a recording medium including instructions executable by a computer, such as a program module executed by the computer. The computer-readable medium may be any available medium that can be accessed by a computer and may include volatile and non-volatile media, and removable and non-removable media. Also, the computer-readable medium may include a computer storage medium and a communication medium. Computer storage media includes volatile and non-volatile, and removable and non-removable media, which are embodied in any method or technology for storage of information such as computer readable instructions, data structures, program modules, or other data. Communication media typically may include computer readable instructions, data structures, other data of modulated data signals, such as program modules, or other transmission mechanisms, and may include any information delivery media.

The term “unit” as used herein may be a hardware component such as a processor or a circuit, and/or a software component executed by a hardware component such as a processor.

The above description of the present specification is for illustration, and it would be understood by those skilled in the art that the description of the present specification can be easily modified into other specific forms without changing the technical concept or essential features of the present disclosure. Therefore, it should be understood that the embodiments described above are exemplary in all aspects and are not limited thereto. For example, each component described in a single form may be implemented in a distributed manner, and similarly, components described in a distributed manner may be implemented in a combined manner.

The scope of the present embodiment is defined by the claims described below rather than the detailed description, and it should be interpreted that all changes or modified forms derived from the meaning and scope of the claims and the equivalent concept thereof are included.

What is claimed is:

1. A method of providing an infertility-associated service, the method comprising:
receiving, from a user terminal, a fertility test request including user information and selection information;
selecting a specimen collection method according to the selection information and performing sampling scheduling for the selected specimen collection method;
receiving a result of a fertility test performed using a specimen collected according to the selected specimen collection method; and
providing the received result of the fertility test to the user terminal.

2. The method of claim 1, wherein the specimen collection method is any one of a specialist visit collection method, a test kit delivery method, or a hospital visit collection method.

3. The method of claim 2, wherein the sampling scheduling for the selected specimen collection method comprises, in a case where the test kit delivery method is selected:
making a delivery company delivery reservation to deliver the test kit to a location of the user included in the user information; and
making a delivery company collection reservation to collect a test kit including the obtained specimen at a location of a user.

4. The method of claim 2, further comprising:
in a case where the test kit delivery method is selected, providing the user terminal with a specimen collection method using the test kit.

5. The method of claim 2, wherein the sampling scheduling for the selected sample collection method comprises:
receiving a completion event in which the delivery of the test kit to a location of a user is completed or the collection of a specimen is completed according to an input of the user; and
making a delivery company collection reservation within a preset period from a time when the completion event occurs.

6. A server for providing an infertility-associated service, the server comprising:
a memory storing at least one least one program;
a communication unit configured to transmit data to and receive data from a user terminal, a delivery company for delivering and collecting test kits, and an examination institution for performing a fertility test; and
at least one processor configured to provide an infertility-associated service to a user, the at least one processor, when executed by the at least one program, configured to;
receive, from the user terminal, a fertility test request including user information and selection information,
select a specimen collection method according to the selection information and perform sampling scheduling for the selected specimen collection method,
receive, from the examination institution, a result of a fertility test performed using the collected specimen, and
provide the received result of the fertility test to the user terminal.

7. A non-transitory computer-readable recording medium storing instructions to perform a method of providing an infertility-associated service, the method comprising:
receiving, from a user terminal, a fertility test request including user information and selection information;
selecting, at a processor, a specimen collection method according to the selection information and performing sampling scheduling for the selected specimen collection method;
receiving, at the processor, a result of a fertility test performed using a specimen collected according to the selected specimen collection method; and
providing, at the processor, the received result of the fertility test to the user terminal.
